(19) United States
(12) Patent Application Publication
(10) Pub. No.: US 2023/0320961 A1
(43) Pub. Date: Oct. 12, 2023

(54) Personal Care Composition Containing a Biosurfactant

(71) Applicant: The Procter & Gamble Company, Cincinnati, OH (US)

(72) Inventors: ISOKEN OMOSEFE IGWEKALANWEKE, Springdale, OH (US); Howard David Hutton, III, Oregonia, OH (US); Alan Glyn Davies, Cincinnati, OH (US)

(21) Appl. No.: 18/298,435

(22) Filed: Apr. 11, 2023

Related U.S. Application Data
(60) Provisional application No. 63/329,743, filed on Apr. 11, 2022.

(51) Int. Cl. A61K 8/42; A61K 8/30; A61K 8/04; A61K 8/72

(52) U.S. Cl. CPC A61K 8/42; A61K 8/30; A61K 8/042; A61K 8/72; A61K 2800/5424; A61K 2800/5428; A61K 2800/30

(57) ABSTRACT

A mild personal care composition that contains an effective amount of a glycolipid biosurfactant, an amphoteric surfactant and is free of thickeners. The glycolipid biosurfactant may be a rhamnolipid or a sophorolipid surfactant that provides good cleansing properties and exhibits consumer desired foam properties. The glycolipid surfactant may be a mixture of sophorolipid surfactants having a hydrophilic/lipophilic balance (HLB) of about 2 to about 7 and an HLB of about 15 to about 20. The composition also includes a sulfate-free anionic surfactant, an amphoteric surfactant and a dermatologically acceptable carrier.

### FIELD OF THE INVENTION

The present disclosure generally relates to sulfate-free personal care compositions that are that contain a glycolipid surfactant. More specifically, the present disclosure relates to sulfate-free personal care compositions containing a mix of high HLB and low HLB sophorolipid surfactants and/or a rhamnolipid surfactant, and which are free of thickeners.

### BACKGROUND OF THE INVENTION

Human hair becomes soiled due to contact with the surrounding environment and from sebum secreted by the scalp. Soiled hair can have an undesirable feel and/or appearance. As a result, people may clean their hair with a shampoo composition that restores the hair to a clean and attractive appearance. Many conventional shampoos use sulfated surfactants such as sodium lauryl sulfate and/or sodium laureth sulfate as a detersive surfactant to clean hair. Sulfated surfactants are generally good at removing oil and other contaminants from hair, but they are sometimes associated with poor quality hair feel, hair dryness and/or skin dryness after washing. This is commonly referred to as “harshness,” and harsh shampoos can face poor consumer acceptance.

Removing sulfated surfactants provides a milder cleansing composition, but there can be drawbacks, especially for conditioning shampoos (i.e., shampoos that provide a cleansing and conditioning benefit to hair). Conditioning shampoos commonly use cationic conditioning polymers to form a coacervate with an anionic surfactant during use, which deposits on hair to provide the easier wet-combing and detangling benefit desired by the user. However, the formation of coacervate ties up a portion of the surfactant within the coacervate and therefore decreases the amount of surfactant available to provide foaming and cleansing.

Sulfate free surfactants, such as isethionate-based surfactants, are sometimes used to avoid some of the perceived harshness of sulfated surfactants. For example, Indian patent application No. 3544/DEL/2015 discloses that sodium cocoyl isethionate (SCI) is the primary sulfate free surfactant of choice for its mildness towards hair and skin. However, non-sulfated surfactants tend to be less effective than sulfated surfactants at foaming and cleansing, and the addition of a cationic polymer can further tax a sulfate-free surfactant system, resulting in further decreases in foaming and cleansing. In addition, SCI may not be suitable for use at an acidic pH (e.g., <pH 6) because it is susceptible to hydrolysis, which also results in decreased foaming, cleaning and stability.

In addition to increased mildness, there is also a desire to provide more environmentally friendly personal cleansing compositions. Surfactants used in conventional cleansing compositions are often derived from petrochemicals that are perceived as being environmentally unfriendly. Yet, sustainability in the use of cosmetic ingredients is becoming increasingly important and something that a growing number of consumers and manufacturers of cosmetic cleansing agents are demanding. The use of certain sustainable or natural-derived surfactants is known. However, naturally derived surfactants are sometimes associated with poor foaming and cleaning performance. In addition, the use of naturally derived surfactants may introduce instability into the composition.

Personal care compositions with a sulfate-free surfactant system commonly include thickeners (e.g., water soluble polymers and inorganic salts) to increase the viscosity of the composition. However, these added thickeners can negatively impact other key performance attributes such as product spreading, quickness of lather generation, and product feel during rinsing. Added thickeners can also make it more difficult to deliver a foamable version of the composition, for example, via an aerosol device or pump.

Accordingly, it would be desirable to provide a personal cleansing composition with a mild surfactant system that includes a naturally derived surfactant that has good foaming and cleaning properties. It would also be desirable for such a composition to be free of sulfated surfactants. It would further be desirable for such a composition to provide good hair conditioning. It would also be desirable to formulate such a composition without the use of rheology modifiers and thickeners that negatively impact other key performance attributes such as product spreading, quickness of lather generation, feel of product during rinsing, etc. The absence of thickeners and low product viscosity could also be advantageous, for example, to deliver a foamable version of the composition via an aerosol device or pump.

### SUMMARY OF THE INVENTION

Disclosed herein are personal care compositions comprising a glycolipid surfactant, a non-sulfate anionic surfactant, an amphoteric surfactant. The compositions are free of thickeners and have a pH of 5.8-7.2. In some aspects, the compositions are free of and comprise a first sophorolipid surfactant having a hydrophilic/lipophilic balance (HLB) of 2 to 7, a second sophorolipid surfactant having an HLB of 15 to 20.

### DETAILED DESCRIPTION OF THE INVENTION

Shampoos containing sulfated surfactants are generally recognized as providing desirable cleansing properties, including sebum and soil removal and good lathering. However, sulfated surfactants are commonly perceived as being harsh. It has now been discovered that replacing the sulfated surfactant(s) in personal cleansing composition with glycolipid surfactants, alone or with other non-sulfated surfactants, can provide desirable cleansing properties without the harshness associated with sulfated surfactants.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition and are based on level of the subject ingredient, which does not include carriers or by-products that may be included in commercially available materials, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

Definitions

“About” modifies a particular value by referring to a range of plus or minus 20% or less of the stated value (e.g., plus or minus 15% or less, 10% or less, or even 5% or less).

“Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Gel network phase” or “dispersed gel network phase” refers to a lamellar or vesicular solid crystalline phase that includes at least one fatty alcohol, at least one gel network surfactant, and a liquid carrier. The lamellar or vesicular phase can be formed of alternating layers with one layer including the fatty alcohol and the gel network surfactant and the other layer formed of the liquid carrier.

“Solid crystalline” refers to the crystalline structure of the lamellar or vesicular phase at ambient temperatures caused by the phase being below its melt transition temperature. For example, the melt transition temperature of the lamellar or vesicular phase may be about 30° C. or more (i.e., slightly above about room temperature). The melt transition temperature can be measured through differential scanning calorimetry, which is conventional measurement method known to those skilled in the art.

“Suitable for application to human hair” means that the personal care composition or components thereof, are acceptable for use in contact with human hair and the scalp and skin without undue toxicity, incompatibility, instability, allergic response, and the like.

“Substantially free of” means a composition or ingredient comprises less than 3% of a subject material (e.g., less than 2%, 1%, or even less than 0.5%), by weight of the composition or ingredient. “Free of” means a composition of ingredient comprises 0% of a subject material.

“Sulfate surfactants” means surfactants that contain a sulfate moiety. Some non-limiting examples of sulfate surfactants are sodium lauryl sulfate, sodium laureth sulfate, ammonium lauryl sulfate and ammonium laureth sulfate.

Personal Care Composition

The personal care compositions herein include a glycolipid surfactant, an amphoteric surfactant, a non-sulfate anionic surfactant and, optionally, other ingredients commonly found in compositions of the type described. Isethionate (e.g., and/or taurate anionic surfactants (e.g., sodium cocoyl isethionate, sodium cocoyl taurate or sodium methyl cocoyl taurate) may be particularly suitable as a non-sulfate anionic surfactant. The personal care compositions herein are free of thickeners and, in some instances, free of sarcosinate surfactants (e.g., sodium lauroyl sarcosinate, sodium myristoyl sarcosinate, potassium myristoyl sarcosinate, sodium cocoyl sarcosinate, sodium oleyl sarcosinate, ammonium oleyl sarcosinate and triethanolamine lauroyl sarcosinate).

The personal care compositions herein may be provided in various product forms such as solutions, suspensions, shampoos, conditioners, lotions, creams, gels, toners, sticks, sprays, aerosols, ointments, cleansing liquid washes, solid bars, pastes, foams, mousses, shaving creams, wipes, strips, patches, hydrogels, film-forming products, facial and skin masks (with and without insoluble sheet), and the like. The composition form may follow from the particular dermatologically acceptable carrier chosen. In some aspects, the personal care compositions described herein may include a dispersed gel network phase that provides a milder, but effective, cleansing benefit to soiled hair in combination with a detersive glycolipid surfactant.

In some aspects, the compositions herein are free of or substantially free of thickeners. For example, the composition may contain less than 1% (e.g., 0% to 0.8%, 0.05% to 0.5%, or even 0.1% to 0.3%) of an inorganic salt thickener such as sodium chloride, potassium chloride, sodium sulfate, ammonium chloride, sodium bromide, combinations of these and the like. In sulfate-free cleansing compositions, inorganic salt can introduce instability to the composition by aiding in the formation of an undesirable coacervate between anionic surfactants and cationic polymers prior to the intended use of the composition. Inorganic salt and other thickeners can undesirably impact the rheological and performance properties of the present composition as well as the consumer-perceived quality of the product.

Other examples of thickeners that may be excluded or substantially excluded from the present compositions include homopolymers based on acrylic acid, methacrylic acid or other related derivatives (e.g., polyacrylate, polymethacrylate, polyethylacrylate, and polyacrylamide), acrylic copolymers or methacrylate copolymers (e.g., acrylates/C10-C30 alkyl acrylate crosspolymer), crosslinked acrylic polymers (e.g., carbomer), hydrophobically modified cellulose derivatives; hydrophobically modified, alkali swellable emulsions (e.g., hydrophobically modified polyacrylates, polyacrylic acids, polyacrylamides and polyethers) cellulose and its derivatives (e.g., microcrystalline cellulose, carboxymethylcelluloses, methylcellulose, ethylcellulose), guar and its derivatives (e.g., hydroxypropyl guar, and hydroxypropyl guar hydroxypropyl trimonium chloride), polyethylene oxide, polypropylene oxide, polyvinylpyrrolidone, polyvinyalcohol and its derivatives, polyethyleneimine and its derivatives, silicas (e.g., fumed silica, precipitated silica, and silicone-surface treated silica), water swellable natural polymers (e.g., xanthan gum, guar gum, arabia gum, carob gum and locust bean gum), sorbitol, karaggenan, pectin, agar, starch (from rice, corn, potato, wheat, etc) and starch derivatives (e.g. carboxymethyl starch, methylhydroxypropyl starch). Still further examples of thickeners, rheology modifiers and suspending agents that may be excluded from the present composition are disclosed in U.S. Pat. No. 10,258,555.

It is to be appreciated that embodiments in which the present compositions include materials commonly used as rheology modifiers (thickeners, etc.) are contemplated herein. In such embodiments the material may be included to provide a function or benefit other than thickening or may be specifically selected because it does not undesirably interact with other ingredients in the composition (form coacervate prior to use, etc.).

Glycolipid Surfactant

The personal care compositions described herein include one or more detersive glycolipid surfactants to help provide a cleaning benefit to a target bodily surface such as soiled hair and skin. Glycolipid surfactants are biosurfactants naturally produced by certain microorganisms and generally consist of a carbohydrate moiety (head) linked to a fatty acid (tail). Without being limited by theory, glycolipid surfactant(s) facilitate cleaning due to their amphiphilic nature, which allows the surfactants to break up, and form micelles around, oil and other contaminants in the hair, which can then be rinsed off with water. Glycolipid surfactants are generally considered milder than sulfate-based surfactants. The glycolipid surfactants suitable for use herein can be described according to the following general formulas, including their salts.

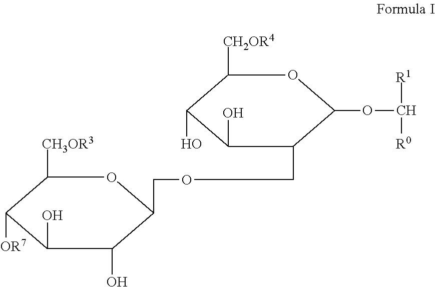

### Where:

R³and R⁴ are independently from each other H or an acetyl group;
R⁵ is H or a saturated or unsaturated, hydroxylated or non-hydroxylated hydrocarbon group having 1 to 9 carbon atoms
R⁶ is the group R⁸—CO—R⁹ or the group:

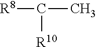

R⁸ is a saturated or an unsaturated, hydroxylated or non-hydroxylated, linear or branched hydrocarbon group having 1 to 20 carbon atoms, the total number of carbon atoms in the groups R⁵ and R⁸ not exceeding 20;
R⁹ is CH₃ or OH or cation salt thereof, or an O(CH₂)ₘCH₃ ester thereof with m being an integer between 0 and 3, or a lactone ring formed with R7;
R¹⁰ is H or OH; and
R⁷ is H or a lactone ring formed with R⁹.

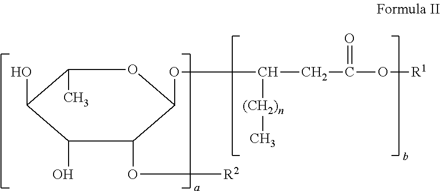

Where: a and b independently from each other are 1 or 2, n is an integer from 4 to 10, R¹ is H or a cation, R² is H or the group

CH₃(CH₂)ₘCH═CH—CO

m being an integer from 4 to 10, and m and n may be the same or different.

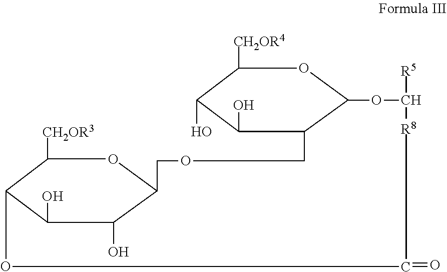

Where: R³, R⁴, R⁵ and R⁸ are as defined above, and at least one of R³ and R⁴ is an acetyl group.

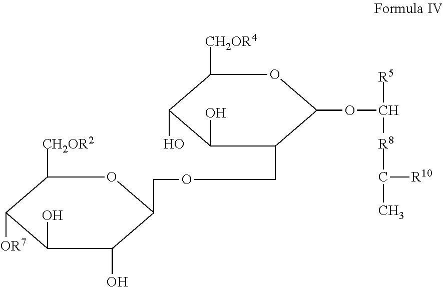

Where: R⁵ is CH₃, R₁₀ is H and R⁸ is a saturated linear hydrocarbon chain having 8-10 carbon atoms.

Glycolipid surfactants suitable for use herein may be selected from rhamnolipids, sophorolipids, glucoselipids, celluloselipids, trehaloselipids, salts of these and combinations thereof. Some non-limiting examples of rhamnolipids and sophorolipids that may be suitable for use herein are disclosed in U.S. Publication No. 2004/0152613; U.S. Pat. No. 9,271,908; and PCT Publication No. WO 2020/016097. Commercially available glycolipids that may be suitable for use herein include FERMA SH and FERMA SL brand sophorolipid surfactants from Locus Performance Ingredients, SPECBIO SL brand sophorolipid surfactant from Spec-Chem Industry, Inc., and RHEANCE ONE brand rhamnolipid surfactant from Evonik. The glycolipid surfactant may be present in the compositions herein at 0.1% to 10% (e.g., 0.5% or even 1% to 5%).

Co-Surfactants

The personal care composition herein may optionally include a co-surfactant selected from anionic surfactants, amphoteric surfactants, zwitterionic surfactants, non-ionic surfactants and combinations of these. Some non-limiting examples of anionic surfactants include non-sulfated anionic surfactants such as isethionates, carboxylates, sulfonates (e.g., alpha olefin sulfonates, linear alkylbenzene sulfonates, alkyl glyceryl sulfonates, sodium laurylglucosides hydroxypropylsulfonate), sulfosuccinates, sulfoacetates, sulfolaurates, amino acid-based surfactants (e.g., glycinates, taurates, alaninates, glutamates), lactate- and lactylate-based surfactants (e.g., sodium lauroyl lactate and sodium lauroyl lactalyte), phosphate ester surfactants and combinations thereof.

Some non-limiting examples of amphoteric and/or zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. For example, amphoacetates, amphodiacetates, betaines, amidobetaines (e.g., cocamidopropyl betaine and lauramidopropyl betaine), amidosulfobetaines, propionates, sultaines, hydroxysultaines, and combinations thereof.

Some non-limiting examples of non-ionic surfactants include glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides acyl glucamides, amine oxides and combinations thereolf. Some particularly suitable examples of non-ionic surfactants include cocamide, cocamide MEA, PPG-2 cocamide, PPG-2 hydroxyethyl cocamide, PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof.

More specific examples of the optional co-surfactants described above are disclosed in US2019/0105246, US2018/0098923, U.S. Pat. No. 9,271,908, WO2020/016097, and McCutcheon's Emulsifiers and Detergents, 2019, MC Publishing Co.

The optional additional surfactants, when present, may be included in the personal care compositions to provide the desired cleaning and lather performance. Any additional surfactants should be physically and chemically compatible with the other components of the personal care compositions described herein and should not otherwise unduly impair product stability, aesthetics, or performance. In some aspects, additional surfactants may be present in the personal care compositions at 5% to 50% (e.g., 8% to 30%, 9% to 25%, or even 10% to 17%).

Dispersed Gel Network Phase

The personal care compositions described herein may include a dispersed gel network phase to cleaning and/or conditioning benefit to the composition in combination with the detersive surfactant. A gel network phase can confer a cleaning benefit to the personal care composition through its hydrophobic nature. Specifically, it is believed, without being limited by theory, that the hydrophobic nature of the dispersed gel network allows the gel network to dissolve hydrophobic soils such as oil into the gel network. Once the soils are dissolved into the gel network, the gel network can be rinsed out of the hair or skin.

Suitable dispersed gel networks can be formed by combining a fatty alcohol and a gel network surfactant in a suitable ratio and heating the dispersion to a temperature above the melting point of the fatty alcohol. During the mixing process, the fatty alcohol melts allowing the gel network surfactant to partition and bring water into the fatty alcohol. Mixing of the gel network surfactant and fatty alcohols also changes the isotropic fatty alcohol drops into liquid crystalline phase drops. When the mixture is subsequently cooled below the melt transition temperature of the fatty alcohols, the liquid crystal phase is converted into a solid crystalline gel network. Additional details of suitable gel networks are described in G. M. Eccleston, “Functions of Mixed Emulsifiers and Emulsifying Waxes in Dermatological Lotions and Creams”, Colloids and Surfaces A: Physiochem. and Eng. Aspects 123-124 (1997) 169-182; and by G. M Eccleston, “The Microstructure of Semisolid Creams”, Pharmacy International, Vol. 7, 63-70 (1986), each of which is incorporated by reference herein.

In some aspects, it may be desirable to pre-form the gel network phase, which means that at least fifty percent of the mixture of the fatty alcohol, gel network surfactant, and liquid carrier are in a substantially solid crystalline phase prior to addition to the other components of the personal care composition. When the dispersed gel network is pre-formed, the gel network component can be prepared as a separate pre-mix, which, after being cooled, can be subsequently incorporated with a detersive surfactant and any other components of a personal care composition. While not intending to be limited by theory, it is believed that incorporation of a pre-formed gel network component with the detersive surfactant and other components of the personal care composition allows the formation of a substantially equilibrated lamellar dispersion (“ELD”) in the final composition.

The ELD is a dispersed lamellar or vesicular phase resulting from the pre-formed gel network component substantially equilibrating with the detersive surfactants, carrier, and other optional components of a personal care composition. This equilibration occurs upon incorporation of the pre-formed gel network component with the other components of a personal care composition and can be effectively complete within about 24 hours after incorporation. The ELD does not form if the components which comprise the gel network component (i.e., the fatty alcohol, the gel network surfactant, and the liquid carrier) are added as individual components together with the other components of the personal care composition in one mixing step, and not as a separate pre-formed gel network component.

The presence of a gel network in the pre-mix and in a personal care composition can be confirmed by means known to one of skill in the art. For example, X-ray analysis, optical microscopy, electron microscopy, and differential scanning calorimetry can be used to identify a gel network. A suitable x-ray analysis method is described in U.S. Publication No. 2006/0024256.

In some aspects, the scale size of the dispersed gel network in a personal care composition can range from about 10 nm to about 500 nm (e.g., 0.5 μm to 10 μm or 10 μm to about 150 μm). The scale size distribution of the dispersed gel network in a personal care composition can be measured with a laser light scattering technique using a Horiba model LA 910 Laser Scattering Particle Size Distribution Analyzer (Horiba Instruments, Inc. Irvine California, USA). The scale size distribution in a personal care composition can be measured by combining 1.75 g of the personal care composition with 30 mL of 3% NH₄Cl, 20 mL of 2% Na₂HPO₄·7H₂O, and 10 mL of 1% laureth-7 to form a mixture. This mixture is then stirred for 5 minutes. As appropriate for the individual Horiba instrument being used, samples in the range of 1 to 40 mL are taken and then injected into the Horiba instrument, which contains 75 mL of 3% NH₄Cl, 50 mL of 2% Na₂HPO₄·7H₂O, and 25 mL of 1% laureth-7, until the Horiba instrument reading is between 88-92% T, which is needed for the scale size measurement. Once this is achieved, a measurement is taken after 2 minutes of circulation through the Horiba instrument to provide the scale size measurement. A subsequent measurement is taken using a sample of the personal care composition which has been heated above the melt transition temperature of all fatty materials present in the shampoo composition to ensure the dispersed gel network is melted. This subsequent measurement allows a scale size distribution to be taken of all of the remaining materials in the personal care composition, which then can be compared to the scale size distribution of the first sample and assist in the analysis.

Gel Network Fatty Alcohol

The dispersed gel network may include a fatty alcohol (e.g., C10-C40 fatty alcohols) at 0.05% or more by weight of the composition (e.g., 0.05% to about 25%, 0.5% to 20%, or 1% to 8%). The fatty alcohol may be straight or branched chain and can be saturated or unsaturated. As can be appreciated, suitable fatty alcohols can be of natural, vegetable, or synthetic origin. In some aspects, it may be desirable to mix several fatty alcohols to provide a dispersed gel network phase with a melt transition temperature of about 38° C. or greater such as, for example, a mixture of cetyl alcohol and stearyl alcohol at a ratio of between 20:80 and 80:20. Some non-limiting examples of fatty alcohols that may be suitable for use herein include cetyl alcohol, stearyl alcohol, arachidyl alcohol, behenyl alcohol, C₂₁ fatty alcohol (1-heneicosanol), C₂₃ fatty alcohol (1-tricosanol), C₂₄ fatty alcohol (lignoceryl alcohol, 1-tetracosanol), C₂₆ fatty alcohol (1-hexacosanol), C₂₈ fatty alcohol (1-octacosanol), C₃₀ fatty alcohol (1-triacontanol), C₂₀₋₄₀ alcohols (e.g., Performacol® 350 and 425 Alcohols, available from New Phase Technologies), C₃₀₋₅₀ alcohols (e.g., Performacol® 550 Alcohol), C₄₀₋₆₀ alcohols (e.g., Performacol® 700 Alcohol), and mixtures thereof.

Gel Network Surfactant

The gel network phase may include a gel network surfactant at 0.01% to 15% by weight of the composition (e.g., 0.1% to about 10%, 0.2% to about 5%). The gel network surfactant is combined with the fatty alcohol and liquid carrier to form a gel network pre-mix, which can then be added to the other ingredients of the personal care composition.

In some aspects, the total weight of the gel network surfactant and the fatty alcohols is 0.5% to about 15% by weight of the personal care composition (e.g., 1% to 10%). In some aspects, the gel network surfactant may be included in the gel network at a desired weight ratio with respect to the fatty alcohols. For example, the ratio of the fatty alcohols to the gel network surfactant may be 1:5 to 100:1 (e.g., 1:1 to 40:1, 2:1 to 20:1, or even 3:1 to 10:1).

The gel network surfactant can be any suitable anionic, zwitterionic, amphoteric, cationic, and nonionic surfactants that is substantially free of sulfates. The detersive surfactant and the gel network surfactant can be the same or different. In some aspects, the gel network surfactant has a hydrophobic tail group with a chain length of 10 to 40 carbon atoms. The hydrophobic tail group may be alkyl, alkenyl (containing up to 3 double bonds), alkyl aromatic, or branched alkyl. Mixtures of more than one gel network surfactant can also be used. Some non-limiting examples of gel network surfactants are disclosed in U.S. Patent App. Pub. No. 2006/0024256.

Liquid Carrier for the Gel Network Phase

In some aspects, the dispersed gel network phase may include a suitable liquid carrier at 0.05% to 95% by weight of the personal care composition. The liquid carrier can be water or another suitable solvent. The carrier and the gel network surfactant may be selected to work together to swell the fatty alcohol, which leads to the formation and stability of the gel network phase. A suitable solvent is any that can be used in the place of or in combination with water in the formation of the gel network phase. In some aspects, the liquid carrier can be substantially free of solvents other than water. In some aspects, the liquid carrier for the dispersed gel network phase can be included at a weight ratio of about 1:1 with the fatty alcohol of the dispersed gel network phase.

Cationic Polymer

The personal care compositions herein may include 0.05%-3% of a cationic polymer (e.g., 0.1-2%, or even 0.2-0.8%) to provide a conditioning benefit (e.g., improved appearance, feel or deposition benefits) to hair or skin. The cationic polymer can have a weight average molecular weight of 100 kDa to 5 MDa (e.g., 500 kDa to 4 MDa, 1 MDa to 3 MDa, or even 1.2 MDa to 2 MDa) and a charge density of 0.4 meq/g to 12 meq/g (e.g., 0.4 to about 2, from about 0.7 to about 2, and from about 0.6 to about 1.6. The charge densities can be measured at the pH of intended use of the personal care composition, which can be pH 3 to pH 9 (e.g., pH 4-8 or pH 4.5-6.5).

The cationic polymer should be selected to form a coacervate with the anionic surfactant, and optional co-surfactant, during the intended use of the personal care composition. The cationic polymers may include cationic, nitrogen-containing moieties such as quaternary ammonium or cationic protonated amino moieties. The cationic protonated amines can be primary, secondary, or tertiary amines, depending on the particular species and the selected pH of the composition. Anionic counterions can be used in association with the cationic polymers, as long as the polymers remain soluble. Examples of suitable counterions include halide counterions (e.g., chloride, fluoride, bromide, iodide). Some nonlimiting examples of cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. Some nonlimiting examples of cationic protonated amino and quaternary ammonium monomers include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of cationic polymers include copolymers of 1-vinyl-2-pyrrolidone and 1-vinyl-3-methylimidazolium salt (referred to in the industry by the Personal Care Products Council (“PCPC”) as polyquaternium-16); copolymers of 1-vinyl-2-pyrrolidone and dimethylaminoethyl methacrylate (polyquaternium-11); cationic diallyl quaternary ammonium-containing polymers, including, for example, dimethyldiallylammonium chloride homopolymer, copolymers of acrylamide and dimethyldiallylammonium chloride (polyquaternium-6 and polyquaternium-7, respectively); amphoteric copolymers of acrylic acid including copolymers of acrylic acid and dimethyldiallylammonium chloride (polyquaternium-22), terpolymers of acrylic acid with dimethyldiallylammonium chloride and acrylamide (polyquaternium-39), and terpolymers of acrylic acid with methacrylamidopropyl trimethylammonium chloride and methylacrylate (polyquaternium-47). In certain embodiments, suitable cationic substituted monomers include cationic substituted dialkylaminoalkyl acrylamides, dialkylaminoalkyl methacrylamides, and combinations thereof.

In certain embodiments, the cationic monomer can be polymethyacrylamidopropyl trimonium chloride, available under the trade name Polycare® 133, from Solvay. In certain embodiment, copolymers of the cationic monomer are also suitable. In such embodiments, the charge density of the total copolymer can be from about 2.0 to about 4.5 meq/gm.

Other cationic polymers include polysaccharide polymers, such as cationic cellulose derivatives and cationic starch derivatives. In certain embodiments, a cationic cellulose polymer can be selected from the salts of hydroxyethyl cellulose reacted with trimethyl ammonium substituted epoxide, referred to as polyquaternium-10 and available from Dow Chemical Company as UCARE™ JR-30M, KG-30 M and LR-30M. Other examples of cationic cellulose polymers include polymeric quaternary ammonium salts of hydroxyethyl cellulose reacted with lauryl dimethyl ammonium-substituted epoxide referred to in the industry (PCPC) as polyquaternium-24.

Further examples of cationic polymers include cationic guar gum derivatives such as the Jaguar® series of guar hydroxypropyltrimonium chloride available from Solvay (Brussels, Belgium) and the N-Hance™ series from Ashland (Wilmington, Delaware). Additional disclosure of cationic guar gum derivatives can be found in U.S. Pat. No. US 6,930,078.

In some instances, the cationic polymer may include a synthetic cationic polymer or derivative thereof present at 0.025% to 5%. Preferred synthetic cationic polymers are generally water-soluble or dispersible and non-crosslinked. In some instances, the synthetic cationic polymer can be a copolymer that includes one or more cationic monomer units and one or more nonionic or anionic monomer units, as long as the copolymer has a net positive charge. Synthetic cationic polymers can have a cationic charge density of 0.5 meq/g to 12 meg/g and an average molecular weight of 1 kDa to 5 MDa. Some non-limiting examples of synthetic cationic polymers are described in U.S. Publication No. US 2003/0223951.

Carrier

The composition may optionally include 20-95% of an aqueous carrier such as water and/or a water miscible solvent. The type and amount of aqueous carrier should be selected to provide the composition with the desired rheological properties. The liquid carrier can be water with, e.g., less than 5%, 3%, 1%, 0.5% or even 0% miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol).

Optional Ingredients

The personal care compositions described herein may include a variety of optional ingredients to tailor the properties and characteristics of the composition, as desired. The optional ingredients may be well known materials that are commonly included in compositions of the type. The options ingredients should be physically and chemically compatible with the essential components of the personal care compositions and should not otherwise unduly impair the stability, aesthetics, or performance of the composition. Individual concentrations of optional components can generally range from about 0.001% to about 10%, by weight of a personal care composition.

Some non-limiting examples of optional ingredients that can be included in the personal care compositions herein include co-surfactants, deposition aids, cationic polymers, conditioning agents (including gel network, triglyceride oils, hydrocarbon oils, fatty esters, silicones), anti-dandruff agents (e.g., zinc pyrithione, zinc carbonate, piroctone olamine, piroctone, ciclopirox, rilopirox, MEA-Hydroxyoctyloxypyridinone, azoxystrobin, sulfur, azoles, salicylic acid and selenium sulfide), anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

Method of Making a Personal Care Composition

The personal care composition described herein can be made using conventional methods for making compositions of the type desired (e.g., shampoo, conditioner or body wash), for example, as described below in the Examples.

In some aspects, the composition may be made by: (a) combining a fatty alcohol, a gel network surfactant, and water at a temperature sufficient to allow partitioning of the secondary surfactant and the water into the fatty alcohol to form a pre-mix; (b) cooling the pre-mix below the chain melt temperature of the fatty alcohol to form a gel network; (c) adding the gel network to one or more detersive surfactants and a liquid carrier to form a personal care composition which includes a dispersed gel network phase having a melt transition temperature of at least about 38° C.

In some aspects, the gel network phase can be prepared by heating the fatty alcohol, the gel network surfactant, and water to a level in the range of about 75° C. to about 90° C. and mixing. This mixture can be cooled to 27-35° C. (e.g., by passing the mixture through a heat exchanger). As a result of this cooling step, at least about fifty percent of the mixture of the fatty alcohol and the gel network surfactant crystallize to form a crystalline gel network.

Other methods of preparing the gel network phase include sonication and/or milling of the fatty alcohol, the gel network surfactant, and water, while these components are heated, to reduce the particle size of the dispersed gel network phase. This results in an increase in surface area of the gel network phase, which allows the gel network surfactant and the water to swell the gel network phase. Another variation in preparing the gel network includes heating and mixing the fatty alcohol and the gel network surfactant first, and then adding that mixture to the water.

In some aspects, the composition may be formulated to have a low viscosity (e.g., less than 3,000 mPa-s, 2,500 mPa-s, 2,000 mPa-s, or even less than 1,000 mPa-s) and placed in a suitable container with a foaming agent. The container may include a pump, valve or other suitable dispensing device that enables to user to dispense the composition as a foam or gel. The foaming agent may be present at 1% to 10% (e.g., 2% to 9% or 3% to 8%), by weight of the composition. In some aspects, the foaming agent can be a propellant. Some non-limiting examples of foaming agents that may be suitable for use herein include chlorofluorocarbons (CFCs) such as dichlorodifluoromethane, 1,1-dichloro-1,1,2,2-tetrafluoroethane, 1-chloro-1,1-difluoro-2,2-trifluoroethane, 1-chloro-1,1-difluoroethylene, monochlorodifluoromethane and mixtures thereof; hydrofluorocarbons (HFCs) such as 1,1-difluoroethane, 1,3,3,3-tetrafluoropropene, 2,3,3,3-tetrafluoropropene and mixtures thereof; hydrofluoroolefins (HFOs) such as 2,3,3,3-tetrafluoropropene (HFO-1234yf), 1,3,3,3-tetrafluoropropene (HFO-1234ze), and mixtures thereof. In addition, the foaming agent/propellants previously can be mixed with one or more hydrocarbons, chlorofluorocarbons, hydrofluorocarbons, hydrofluoroolefins, alkyl ethers, and compressed gases.

Method of Use

The personal care compositions described herein can be used in a conventional manner for cleansing and conditioning of hair or skin. Effective amounts of the composition for use generally range from 1 g to 50 g (e.g., 1 g to about 20 g). Generally, a method of treating hair or skin can include applying the personal care composition to the hair or skin. For example, an effective amount of the personal care composition can be applied to the hair or skin, which has been wetted with water, and then the composition can be rinsed off. Application to the hair typically includes working the composition through the hair such that most or all of the hair is contacted with the composition. The personal care composition can be used as a liquid solid, semi-solid, flake, gel or foamin a pressurized container with a propellant added, or used in a pump spray form. The viscosity of the product may be selected to accommodate the form desired.

In some aspects, the method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

### METHODS

Blender Lather Height

Consumers commonly associate lathering with the quality of a personal cleansing composition such as a shampoo. This method provides a way to simulate the lather produced by surfactants when used on hair under typical shampooing conditions and quantify certain lather properties. Oil (e.g., sebum) is one of the most common contaminates found on hair that can undesirably affect the lather properties of a shampoo. Thus, this method evaluates the effect of oil on lather properties.

100 mL of tap water, which has a water hardness of about 6-8 gpg (at 100 oF), is added to a suitable blender (e.g., KitchenAid KSB560CU1 brand food mixer or equivalent), followed by 2 mL of the test composition and 1 mL of extra virgin olive oil. Blend the mixture on “stir” setting for 30 seconds and measure the height of the lather within the blender in centimeters and record as Lather Height.

In some instances, it may be desirable to calculate the Lather Height to be expected from a mixture of glycolipid surfactants, based on the weight average Lather Height of the individual surfactants. At a constant co-surfactant concentration, the weighted average Lather Height of a composition containing a mixture of high HLB glycolipid surfactant and low HLB glycolipid surfactant can be calculated using the following equation:

weighted average Lather Height=(w_a X_a+w_b X_b)/(w_a+w_b)
Where:
w_a=wt % of high HLB surfactant in the glycolipid surfactant mixture;
X_a=Lather Height of high HLB only composition;
w_b=wt % of low HLB surfactant in the glycolipid surfactant mixture; and
X_a=Lather Height of the low HLB surfactant only composition.

Wet Hair ILS Protocol

This method can be used to determine the cleaning and/or conditioning properties of the personal care compositions herein. In this method, a 20 g hair switch of Caucasian Low Lift Hair Tresses (e.g., from International Hair Importers and Products, Inc., Glendale, NY) is wetted with water, treated with a personal care composition and subject to testing in an In-Lab Screening (ILS) sink. The ILS sink has a salon spray head/hose that is held in place but can be directed to run water over a hair tress that hangs from a rod placed over the sink. The tress can be moved in and out of the water as necessary. The water is maintained at a temperature of 38° C. and a flow rate of 5.7 liters per minute. The testing is as follows:
Calibrate ILS sink to 38° C.
Hang the hair tress switch on rod in sink.
Wet hair thoroughly for 30 seconds. Squeegee the hair tress switch once using your index and middle finger (“scissor fingers) from top to bottom to remove excess water.
Apply 0.1 g/g (product/hair) of the test composition to the front of the switch, from top to bottom.
Milk the hair tress switch for 15 seconds by stroking the tress from the top to the bottom while alternating hands to create lather. Flip the bottom of the switch to the top and milk for another 15 seconds.
Evaluate Lather Creaminess (look and feel, after 30 seconds of lathering). Scale: 0=No Creaminess-10=Extremely Creamy
Evaluate Lather Combing (with lather still in switch). Using the lowest pressure possible, place comb all the way through the hair (starting at the top) and using a minimal amount of force (comb from top to bottom) comb through hair switch. Scale: 0=hard to comb-10=easy to comb.
Rinse for 30 seconds (while lightly milking the switch).
Squeegee the hair switch tress once with scissor fingers.
Evaluate Slippery Feel. Squeegee once and assess feel. Scale: 0=No Slip-10=Extremely Slippery.
Evaluate Clean Feel Post Rinse. Stroke the hair from top to bottom between the thumb and two fingers with medium pressure. Gauge how clean or dirty the hair feels. Scale: 0=Low (Dirty)-10=High (Clean).
Evaluate Post Rinse Comb. Using the lowest pressure possible, placing comb (wide tooth side) all the way through the hair (front to back), and using a minimal amount of force (top to bottom) comb through hair switch. Scale: 0=Hard-10=Easy.

### Viscosity Method

Composition viscosities herein are measured on a 2.5 mL sample using a cone and plate Brookfield® RS brand rheometer with cone C75-1 at 2 s-1, 27° C. at 3 mins.

### EXAMPLES

### Example 1: Comparative and Inventive Compositions

Tables 1A and 1B show a comparative composition (C1) along with examples of inventive compositions. The comparative composition is made using conventional methods of making shampoo compositions. The inventive compositions are made according to the processes described below.

For examples 1-6, DI water is added to a mixing vessel and heated to 75° C.±3° C. while agitating. Sodium cocoyl isethionate (SCI) is added to the mixing vessel and mixed until the SCI has fully dissolved (i.e., no visible particles remain, and the batch is clear). After the SCI has fully dissolved, the following materials are added to the mixing vessel: sodium benzoate, tetrasodium EDTA, sodium salicylate, alkyl amidopropyl betaine, and glycolipids. The vessel contents are mixed for at least 10 minutes, and then cooled to less than 35° C. A polyquaternium-10 slurry is made with water, which is then immediately added to the mixing vessel and mixed for 10 minutes. Optionally, perfume may be added and mixed in the mixture for at least 2 minutes. Citric acid is used to titrate the mixture until a pH of 5.5 to 5.8 is reached. DI water is added to bring the final volume to 100%. The mixture is mixed for at least 10 minutes until homogeneity is achieved.

For examples 7-8, DI water is added to a mixing vessel. While agitating, the following materials are added to the mixing vessel: KATHON brand methylchloroisothiazolinone and methylisothiazolinone and the glycolipid surfactant. The vessel contents are mixed for at least 10 minutes. Citric acid is used to titrate the mixture until a pH of 5.5 to 5.8 is reached. DI water is added to bring the final volume to 100%. The mixture is mixed for at least 10 minutes until homogeneity is achieved.

For examples 9-12, DI water is added to a mixing vessel and heated to 75° C.±3° C. while agitating. Sodium cocoyl isethionate (SCI) is added to the mixing vessel, and the mixing continues until the SCI has fully dissolved (with no visible particles remaining and batch is clear). After the SCI has fully dissolved, the following materials are added to the mixing vessel: sodium benzoate, tetrasodium EDTA, sodium salicylate, alkyl amidopropyl betaine and the glycolipid surfactant(s). The vessel contents are mixed for at least 10 minutes. The batch is then cooled to less than 35° C. A polyquaternium-10 slurry is made with water, which is immediately added to the mixing vessel and mixed for 10 minutes. Perfume is optionally added and mixed for at least 2 minutes. Sodium hydroxide is used to titrate the mixture until a pH of 7.0 to 7.3 is reached. DI water is added to bring the final volume to 100%. The mixture is mixed for at least 10 minutes until homogeneity is achieved.

For examples 13-14, DI water is added to a mixing vessel while agitating. The following materials are then added to the mixing vessel: KATHON brand methylchloroisothiazolinone and methylisothiazolinone and the glycolipid surfactant. The vessel contents are mixed for at least 10 minutes. Sodium Hydroxide is used to titrate the mixture until a pH of 7.0 to 7.3 is reached. DI water is added to bring the final volume to 100%. The mixture is mixed for at least 10 minutes until homogeneity is achieved

TABLE 1A
pH 5.5-5.8

| | C 1 | INV 1 | INV 2 | INV 3 | INV 4 | INV 5 | INV 6 | INV 7 | INV 8 |
|---|---|---|---|---|---|---|---|---|---|
| Lauramidopropyl betaine ¹ | 5.36 | 5.36 | 5.36 | 5.36 | 5.36 | 5.36 | 5.36 | — | — |
| Cocamidopropyl betaine ² | 4.39 | 4.39 | 4.39 | 4.39 | 4.39 | 4.39 | 4.39 | — | — |
| Sodium cocoyl isethionate ³ | 5.1 | 5.1 | 5.1 | 5.1 | 5.1 | 5.1 | 5.1 | — | — |
| Glycolipid (high HLB) ⁴ | — | 0.6 | 0.45 5 | 0.3 | 0.15 | — | 0.2 | 10 | — |
| Glycolipid (low HLB) ⁵ | — | — | 0.15 | 0.3 | 0.45 | 0.6 | 0.2 | — | 10 |
| Glycolipid ⁶ | — | — | — | — | — | — | 0.2 | — | — |
| Polyquaternium-10 ⁷ | 0.25 | 0.25 | 0.25 | 0.25 | 0.25 | 0.25 | 0.25 | — | — |
| Sodium benzoate | 0.75 | 0.75 | 0.75 | 0.75 | 0.75 | 0.75 | 0.75 | — | — |
| Sodium salicylate | 0.45 | 0.45 | 0.45 | 0.45 | 0.45 | 0.45 | 0.45 | — | — |
| Tetrasodium EDTA | 0.16 | 0.16 | 0.16 | 0.16 | 0.16 | 0.16 | 0.16 | — | — |
| Kathon ⁸ | — | — | — | — | — | — | — | 0.03 | 0.03 |
| Perfume | 1.10 | 1.10 | 1.10 | 1.10 | 1.10 | 1.10 | 1.10 | — | — |
| Citric acid | to pH 5.5-5.8 | | | | | | | | |
| Water | QS | | | | | | | | |
| Ratio of glycolipids to | 0:0.6:1 | 0.1:0.6:1 | 0.1:0.6:1 | 0.1:0.6:1 | 0.1:0.6:1 | 0.1:0.6:1 | 0.1:0.6:1 | 1:0:0 | 1:0:0 |
| anionic surfactant to | | | | | | | | | |
| amphoteric surfactant | | | | | | | | | |
| Viscosity (mPa-s) | 3829 | 3743 | 3541 | 3426 | 3357 | 3268 | 3402 | <800 | <800 |
| Lather Combing | 2 | 6 | 5 | 10 | 6 | 4 | 3 | 1 | 2 |
| Slippery Feel | 4 | 6 | 5 | 7 | 8 | 6 | 7 | 0 | 2 |

TABLE 1B
pH 7.0-7.3

| | INV 9 | INV 10 | INV 11 | INV 12 | INV 13 | INV 14 |
|---|---|---|---|---|---|---|
| Lauramidopropyl betaine ¹ | 5.36 | 5.36 | 5.36 | 5.36 | — | — |
| Cocamidopropyl betaine ² | 4.39 | 4.39 | 4.39 | 4.39 | — | — |
| Sodium cocoyl isethionate ³ | 5.1 | 5.1 | 5.1 | 5.1 | — | — |
| Glycolipid (high HLB) ⁴ | 0.6 | 0.45 | 0.3 | — | 10 | — |
| Glycolipid (low HLB) ⁵ | — | 0.15 | 0.3 | 0.6 | — | 10 |
| Glycolipid ⁶ | — | — | — | — | — | — |
| Polyquaternium-10 ⁷ | 0.25 | 0.25 | 0.25 | 0.25 | — | — |
| Sodium benzoate | 0.75 | 0.75 | 0.75 | 0.75 | — | — |
| Sodium salicylate | 0.45 | 0.45 | 0.45 | 0.45 | — | — |
| Tetrasodium EDTA | 0.16 | 0.16 | 0.16 | 0.16 | — | — |
| Kathon ⁸ | — | — | — | — | 0.03 | 0.03 |
| Perfume | 1.10 | 1.10 | 1.10 | 1.10 | — | — |
| Sodium hydroxide | to pH 7.0-7.3 | | | | | |
| Water | QS | | | | | |
| Ratio of glycolipids to | 0.1:0.6:1 | 0.1:0.6:1 | 0.1:0.6:1 | 0.1:0.6:1 | 1:0:0 | 1:0:0 |
| anionic surfactant to | | | | | | |
| amphoteric surfactant | | | | | | |
| Viscosity (mPa-s) | <800 | <800 | <800 | <800 | <800 | <800 |
| Lather Blender Height | 8.0 | 7.0 | 6.0 | 5.5 | 1.0 | 1.3 |
| (cm) | | | | | | |
| Lather Blender Creaminess | 5 | 4 | 3 | 3 | 0 | 0 |
| Rating | | | | | | |
| Lather Combing | 4 | 5 | 9 | 8 | 2 | 1 |
| Ease of Combing | 5 | 4 | 9 | 10 | 2 | 1 |

¹ MACKAM DAB ULS from Solvay
² AMPHOSOL HCA-HP from Stepan
³ HOSTAPON SCI 85 C from Clariant
⁴ FERMA SH brand sophorolipid surfactants from Locus (HLB = 15-20).
⁵ FERMA SL brand sophorolipid surfactants from Locus (HLB = 2-7).
⁶ RHEANCE ONE brand rhamnolipid surfactant from Evonik (HLB 4-13).
⁷ UCARE Polymer JR-30M from Dow (CD = 1.25 meq/g).
⁸ Methylchloroisothiazolinone and methylisothiazolinone from Dupont.

### Example 2: Hair Conditioning

This example demonstrates the hair conditioning benefits provided by the compositions from Table 1A. The combination of a non-sulfate, anionic surfactant such as SCI is commonly thought to provide poor conditioning benefits because the SCI will not form a suitable coacervate during use. The addition of a non-ionic glycolipid surfactant would not be expected to change the conditioning properties of the composition.

The conditioning properties shown in Table 2 below were measured according to the test methods described above.

TABLE 2

| | | | | | | | |
|---|---|---|---|---|---|---|---|
| C 1 | INV 1 | INV 2 | INV 3 | INV 4 | INV 5 | INV 6 | INV 7 |

| | | | | | | | | |
|---|---|---|---|---|---|---|---|---|
| pH | 5.8 | 5.8 | 5.8 | 5.8 | 5.8 | 5.8 | 5.8 | 5.8 |
| Lather Combing rating | 2 | 6 | 5 | 10 | 6 | 4 | 1 | 2 |
| Slippery Feel rating | 4 | 6 | 5 | 7 | 8 | 6 | 0 | 2 |
| Ease of Combing rating | 1 | 6 | 6 | 8 | 8 | 7 | 3 | 3 |
| Viscosity (mPa-s) | 3829 | 3743 | 3541 | 3426 | 3357 | 3268 | <800 | <800 |

Surprisingly, as can be seen in Table 2, the test compositions with a sophorolipid surfactant exhibited improved conditioning properties relative to the control. Even more surprisingly, when the high HLB sophorolipid surfactant and low HLB surfactant are present at a weight ratio of 1:1 (INV 3), the composition appears to synergistically improve the conditioning properties of the composition relative to the control.

### Example 3: Hair Cleaning and Conditioning

This example demonstrates the hair cleaning and conditioning benefits provided by the inventive compositions from Table 1B. The conditioning properties shown in Table 3 below were measured according to the test methods described above.

TABLE 3

| | C 1 | INV 9 | INV 10 | INV 11 | INV 12 | INV 13 | INV 14 |
|---|---|---|---|---|---|---|---|
| pH | 5.8 | 7.0 | 7.0 | 7.0 | 7.0 | 7.0 | 7.0 |
| Lather Blender Height | 5 | 8 | 7 | 6 | 5.5 | 1 | 1.25 |
| Lather Blender Creaminess | 2 | 5 | 4 | 3 | 3 | 0 | 0 |
| Lather Combing rating | 2 | 4 | 5 | 9 | 8 | 2 | 1 |
| Ease of Combing rating | 1 | 5 | 4 | 9 | 10 | 2 | 1 |
| Viscosity (mPa-s) | 3829 | <800 | <800 | <800 | <800 | <800 | <800 |

As can be seen in Table 3, the inventive compositions exhibit improved lathering and conditioning properties relative to the control. This is surprising because the addition of a relatively low level of glycolipid surfactant would generally be expected to have no noticeable impact on these properties. Even more surprising are the much improved cleaning and conditioning properties (Lather Combining and East of Combing) exhibited by compositions that have 50% or more low HLB sophorolipid surfactant (i.e., INV 11 and INV 12).

The data in Table 3 suggest that pH may play an important role in the viscosity of the composition, as well as the cleaning, lathering and/or conditioning properties of the composition. As can be seen in Table 3, all the inventive compositions have a viscosity of less than 800 mPa-s. Thus, it may be possible to use pH to control the viscosity of the composition, thereby providing more formulation and use flexibility for thickener-free compositions.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a first sophorolipid surfactant having a hydrophilic/lipophilic balance (HLB) of about 2 to about 7;
a second sophorolipid surfactant having an HLB of about 15 to about 20;
an anionic surfactant;
an amphoteric surfactant; and
a carrier, wherein the composition is substantially free of thickeners and sulfate surfactants.

2. The personal care composition of claim 1, further comprising a rhamnolipid surfactant.

3. The personal care composition of claim 2, wherein the rhamnolipid surfactant has an HLB of about 4 to about 13.

4. The personal care composition of claim 1, wherein the first and second sophorolipid surfactants are present at a weight ratio of first sophorolipid surfactant to second sophorolipid surfactant of 1:3 to 3:1.

5. The personal care composition of claim 4, wherein the first and second sophorolipid surfactants are present at a weight ratio of 1:1.

6. The personal care composition of claim 1, wherein the anionic surfactant comprises an isethionate surfactant.

7. The personal care composition of claim 6, wherein the composition is free of sarcosinate surfactants.

8. The personal care composition of claim 1, wherein the amphoteric surfactant comprises lauramidopropyl betaine, cocamidopropyl betaine, or a combination thereof.

9. The personal care composition of claim 1, wherein the composition has a pH of about 5.5 to about 7.2.

10. The personal care composition of claim 9, wherein the composition has a pH of about 5.5 to about 5.8.

11. The personal care composition of claim 9, wherein the composition has a pH of about 6.8 to about 7.2.

12. The personal care composition of claim 1, further comprising a dispersed gel network.

13. The personal care composition of claim 12, wherein the dispersed gel network comprises a fatty alcohol, a gel network surfactant, and a liquid carrier, and wherein the fatty alcohol is in the form of liquid crystal drops.

14. The personal care composition of claim 12, wherein the dispersed gel network comprises about 0.05 wt % or more of a fatty alcohol and about 0.01 wt % or more of a gel network surfactant selected from anionic surfactants, cationic surfactants, zwitterionic surfactants, non-ionic surfactants, and mixtures thereof.

15. The personal care composition of claim 1, further comprising a cationic polymer.

16. The personal care composition of claim 1, wherein the composition exhibits a synergistic conditioning benefit according to the Wet Hair ILS method.

17. The personal care composition of claim 1, wherein the composition has a viscosity of less than about 4 Pa-s according to the Viscosity method.

18. A method of cleaning a target body surface, comprising:
wetting the target body surface with water;
applying about 1 to about 50 g of the personal care composition of claim 1 to the wetted body surface;
working the composition on the wetted bodily surface to produce a lather; and
rinsing the composition from the target body surface with water.

19. The personal care composition of claim 18, wherein the first and second sophorolipid surfactants are present at a weight ratio of first sophorolipid surfactant to second sophorolipid surfactant of 1:3 to 3:1.
